(19) United States
(12) Patent Application Publication
(10) Pub. No.: US 2018/0185519 A1
(43) Pub. Date: Jul. 5, 2018

(54) ASSESSMENT OF CORONARY HEART DISEASE WITH CARBON DIOXIDE

(71) Applicant: Cedars-Sinai Medical Center, Los Angeles, CA (US)

(72) Inventors: Rohan Dharmakumar, Moorpark, CA (US); Debiao Li, San Marino, CA (US); Sotirios A. Tsaftaris, Lucca (IT)

(73) Assignee: Cedars-Sinai Medical Center, Los Angeles, CA (US)

(21) Appl. No.: 15/672,162

(22) Filed: Aug. 8, 2017

Related U.S. Application Data
(63) Continuation of application No. 14/075,918, filed on Nov. 8, 2013, now abandoned.
Application No. 14/075,918 is a continuation-in-part of application No. 14/115,860, filed on Nov. 5, 2013.
(60) Provisional application No. 61/482,956, filed on May 5, 2011.

(51) Int. Cl. A61K 49/08; A61K 31/7076; A61K 33/00

(52) U.S. Cl. CPC A61K 49/08; A61K 2300/00; A61K 33/00; A61K 31/7076

(57) ABSTRACT

The invention provides methods for diagnosing coronary heart disease in a subject in need thereof comprising administering an admixture comprising CO₂ to a subject to reach a predetermined PaCO₂ in the subject to induce hyperemia, monitoring vascular reactivity in the subject and diagnosing the presence or absence of coronary heart disease in the subject, wherein decreased vascular reactivity in the subject compared to a control subject is indicative of coronary heart disease. The invention also provides methods for increasing sensitivity and specificity of BOLD MRI.

### GOVERNMENT RIGHTS

The invention was made with government support under Grant No. HL091989 awarded by the National Institutes of Health. The government has certain rights to the invention.

### FIELD OF INVENTION

The invention is directed to methods for detecting coronary heart disease using carbon dioxide (CO₂) to induce hyperemia and monitor vascular reactivity.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Coronary artery disease (CAD) leads to narrowing of the small blood vessels that supply blood and oxygen to the heart. Typically, atherosclerosis is the cause of CAD. As the coronary arteries narrow, blood flow to the heart can slow down or stop, causing, amongst other symptoms, chest pain (stable angina), shortness of breath and/or myocardial infarction. Numerous tests help diagnose CAD. Such tests include coronary angiography/arteriography, CT angiography, echocardiogram, electrocardiogram (ECG), electron-beam computed tomography (EBCT), magnetic resonance angiography, nuclear scan and exercise stress test. Functional assessment of the myocardium (for example the assessment of myocardium's oxygen status) requires that a patient's heart is stressed either via controlled exercise or pharmacologically.

Assessment of vascular reactivity in the heart is the hallmark of stress testing in cardiac imaging aimed at understanding ischemic heart disease. This is routinely done in Nuclear Medicine with radionuclide injection (such as Thallium) in conjunction with exercise to identify territories of the heart muscle that are subtended by a suspected narrowed coronary artery. In patients who are contraindicated for exercise stress-testing, this approach is typically used in conjunction with hyperemia inducing drugs, for example via adenosine infusion. Reduced coronary narrowing is expected to reduce hyperemic response and the perfusion reserve. Since nuclear methods are hampered by the need for radioactive tracers combined with limited imaging resolution, other imaging methods, such as ultrasound (using adenosine along with microbubble contrast) and MRI (also using adenosine and various conjugates of gadolinium (Gd) (first-pass perfusion) or alterations in oxygen saturation in response to hyperemia, also known as the Blood-Oxygen-Level-Dependent (BOLD) effect) are under clinical investigation. Nonetheless, in patients who are contraindicated for exercise stress-testing, currently all imaging approaches require adenosine to elicit hyperemia. However, adenosine has undesirable side effects (such as the feeling of “impending doom”, bradycardia, arrhythmia, transient or prolonged episode of asystole, ventricular fibrillation (rarely), chest pain, headache, dyspnea, and nausea), making it less than favorable for initial or follow-up studies and many patients request that they do not undergo repeated adenosine stress testing. Nonetheless repeated stress testing is indicated in a significant patient population to assess the effectiveness of interventional or medical therapeutic regimens. In view of the side effects of hyperemia inducing drugs, there is a need for alternatives, which induce hyperemia in patients who are contraindicated for exercise stress-testing but do not cause the side effects caused by the existing hyperemia inducing drugs.

### SUMMARY OF THE INVENTION

Applicants' invention is directed to the use of carbon dioxide to replace adenosine to induce hyperemia in subjects contra-indicated for exercise stress testing so as to diagnose coronary heart diseases but without the side effects of adenosine. In an embodiment, the CO₂ levels are altered while the O₂ levels are held constant. In another embodiment, the CO₂ levels are controlled by administering a blend of air and a controlled amount of a gas mixture comprising 20% oxygen and 80% carbon dioxide.

The invention is directed to methods for diagnosing coronary heart disease in a subject in need thereof comprising administering an admixture comprising CO₂ to a subject to produce a hyperemic response corresponding to at least one selected increase in a subject's coronary PaCO₂, monitoring vascular reactivity in the subject and diagnosing the presence or absence of coronary heart disease in the subject. The presence of coronary disease can be detected by monitoring a parameter indicative of a disease-associated change in a vasoreactive response to the at least one increase in PaCO₂ in at least one coronary blood vessel or region of the heart. The inventors have found that such a change can be captured by monitoring the quantum of change in a parameter affected by a change in PaCO₂, from an first PaCO₂ level to a PaCO₂ second level, for example a parameter correlated with vasodilation such as increased blood flow.

An observation of a change in a vasodilatory response can be extended to comparing responses among different subjects, wherein a decreased vascular reactivity in a subject in need of a diagnosis compared to that of a control subject is indicative of coronary heart disease.

Thus, according to one embodiment, the invention also provides a method for assessing hyperemic response in a subject in need thereof comprising administering an admixture comprising CO₂ to a subject to reach a predetermined PaCO₂ in the subject to induce hyperemia, monitoring vascular reactivity in the subject and assessing hyperemic response in the subject, wherein decreased vascular reactivity in the subject compared to a control subject is indicative of poor hyperemic response, thereby assessing hyperemic response in the subject in need thereof.

The invention may be directed to assessing organ perfusion in a subject in need thereof.

The invention may be directed to assessing vascular reactivity of an organ in a subject in need thereof.

The invention further provides methods of producing coronary vasodilation in a subject in need thereof comprising administering an admixture comprising CO₂ to a subject to reach a predetermined PaCO₂ in the subject so as to produce coronary vasodilation, thereby producing coronary vasodilation in the subject.

The invention also provides methods from increasing sensitivity and specificity for BOLD MRI. The method includes administering an admixture comprising CO₂ to a subject to reach a predetermined PaCO₂ in the subject to induce hyperemia and imaging the myocardium using MRI to assess a hypermic response in response to a predetermined modulation in PaCO₂. Optionally, imaging the myocardium comprises (i) obtaining free-breathing cardiac phase resolved 3D myocardial BOLD images; (ii) registering and segmenting the images to obtain the myocardial dynamic volume; and (iii) identifying ischemic territory and quantify image volume.

The invention is also directed to the use a CO2 containing gas for inducing hyperemia in a subject in need of a diagnostic assessment of coronary heart disease, wherein the CO₂ containing gas is used to attain at least one increase in a subject's coronary PaCO₂ sufficient for diagnosing coronary heart disease from imaging data, wherein the imaging data is indicative of a cardiovascular-disease-associated vasoreactive response to the least one increase in PaCO₂ in at least one coronary blood vessel or region of the heart.

The invention also provides a method for inducing hyperemia in a subject in need of a diagnostic assessment of coronary heart disease comprising administering a CO2 containing gas, attaining at least one increase in a subject's coronary PaCO₂ sufficient for diagnosing coronary heart disease from imaging data and imaging the heart during a period in which the increase in PaCO₂ is measurable, wherein the imaging data is indicative of a cardiovascular disease-associated vasoreactive response in at least one coronary blood vessel or region of the heart.

Optionally, the at least one increase in the subject's PaCO₂ is selected to produce a coronary vasoreactive response sufficient for replacing a hyperemia inducing drug in assessing coronary disease.

Optionally, the use/method comprises attaining a particular predetermined PaCO₂.

Optionally, the pre-determined PaCO2 is patient specific, for example an 8 to 20 mm Hg increase relative a baseline steady level measured at the time of testing.

Optionally, the use/method comprises administering carbon dioxide in a stepwise manner.

Optionally, the use/method comprises administering carbon dioxide in a block manner.

Optionally, the CO₂ is administered via inhalation.

Optionally, the disease-associated coronary vasoreactive response is assessed relative to a control subject.

Optionally, the PaCO₂ is increased and decreased repeatedly.

Optionally, the at least one PaCO₂ produces at least an 8%-12% increase in a BOLD signal intensity.

Optionally, the disease-associated vasoreactive response is a compromised increase in blood flow.

Optionally, the imaging data is indicative of the presence or absence of a two-fold increase in blood flow in a coronary artery.

Optionally the imaging data are obtained by MRI and the imaging method obtains input of a change in signal intensity of a BOLD MRI signal.

Optionally, the imaging method is PET or SPECT and the measure of a disease-associated vasoreactive response is the presence or absence of a threshold increase in blood flow.

Optionally, the at least one increase in PaCO₂ produces at least a 10% increase in intensity of a BOLD MRI signal.

Optionally, the at least one increase in PaCO₂ produces a 10-20% increase in intensity of a BOLD MRI signal.

Optionally, the use/method comprises: (i) imaging the myocardium to obtain free-breathing cardiac phase resolved 3D myocardial BOLD images. (ii) registering and segmenting the images to obtain the myocardial dynamic volume and (iii) identifying ischemic territory and quantifying image volume.

Optionally, the at least one PaCO₂ is at least a 10 mm Hg increase from a first level which is determined to be between 30 and 55 mm Hg. Optionally, the first level is first determined to be between 35 and 45 mm Hg.

Optionally, the sufficiency of the increase in PaCO₂ is determined by increasing PaCO₂ in a stepwise manner.

Optionally, the vasoreactive response is sufficient for obtaining a disease-associated change in BOLD MRI signal obtained by administering CO2 in a manner effective to alternate between two or more PaCO₂ levels over a period of time and using repeated BOLD MRI measurements to statistically assess the hyperemic response.

Optionally, the coronary vasoreactive response corresponds to a vasodilatory response produced by administering a hyperemia inducing drug for a duration and in amount per unit of time effective to assess coronary disease.

Optionally, the hyperemia inducing drug is adenosine, wherein adenosine is administered in a regimen of 140 milligrams/litre per minute over 4 to 6 minutes.

Optionally, the use/method comprises admixing air with a selected amount of a CO₂ containing gas controlled to obtain a predetermined size increase in PaCO₂ from a previous value, for example a measured baseline value.

The CO₂ containing gas may contain, for example, 75 to 100% CO₂. Optionally the CO₂ containing gas comprises a percentage composition of oxygen in the 18-23% range, optionally about 20%.

In one embodiment the invention is directed to a method for diagnosing coronary heart disease in a subject in need thereof comprising:
(i) administering an admixture comprising CO₂ to a subject in a stepwise or block manner to reach a predetermined PaCO₂ in the subject to induce hyperemia;
(ii) monitoring vascular reactivity in the subject; and
(iii) diagnosing the presence or absence of coronary heart disease in the subject, wherein decreased vascular reactivity in the subject compared to a control subject is indicative of coronary heart disease,

thereby diagnosing coronary heart disease in the subject in need thereof.

As elaborated below, administering carbon dioxide to alter PaCO₂ in block manner, is optionally repeated over time. Optionally carbon dioxide is administered so as to alternate between two or more levels of PaCO₂ over a period of time.

Vascular reactivity may be monitored using any one or more of a variety of advanced imaging methods including positron emission tomography (PET), single photon emission computed tomography/computed tomography (SPECT), computed tomography (CT), and magnetic resonance imaging (MRI), to name a few. Optionally, vascular reactivity may be measured using FFR.

A particularly advantageous admixture of CO₂ and O₂ for inducing hyperemia, particularly for blending a CO₂ containing gas with air for inhalation is an admixture in which O₂ is present in the range of 19-22%, for example about 20%. In this embodiment, CO2 may make up the rest of the admixture (81-78% respectively) or there may be a third gas in the admixture.

### BRIEF DESCRIPTION OF FIGURES

FIG. 1 depicts, in accordance with an embodiment of the present invention, the vascular reactivity in dogs as measured by the BOLD-effect using medical-grade Carbogen (5% CO₂ and 95% O₂) with and without coronary artery stenosis.

FIG. 2 depicts myocardial BOLD MRI with CO₂ in canines under normocarbic and hypercarbic conditions under free breathing conditions.

FIG. 3 depicts myocardial BOLD response to step-wise PaCO₂ ramp up in canines while holding basal PaO₂ constant.

FIG. 4 depicts myocardial BOLD response to repeated (block) administration CO₂ response.

FIG. 5 depicts the Doppler flow through the left anterior descending artery in response to PaCO₂ modulation while PaO₂ is held constant.

FIG. 6 depicts the Doppler flow through the LAD, RCA and LCX arteries in response to PaCO₂ modulation while PaO₂ is held constant.

FIG. 7 is a bar graph depicting the territorial myocardial BOLD response to PaCO₂ modulations in canines while PaO₂ is held constant.

FIG. 8 is a bar graph depicting the BOLD effect associated with PaCO₂ modulation in blood, muscle and air while PaO₂ is held constant.

FIG. 9 is a table summarizing the statistical BOLD data associated with the PaCO₂ modulation in myocardial territories, blood, muscle and air, while PaO₂ is held constant.

FIG. 10 is a comparison of BOLD response to adenosine and PaCO₂ (while PaO₂ is held constant).

FIG. 11 depicts the early findings of BOLD response to PaCO₂ in humans, while PaO₂ is held constant.

FIG. 12(a) depicts a simulated BOLD signal for a change in PaCO₂ (red line) with definitions for noise variability (σ=20) and response. FIG. 12(b) depicts a relation between BOLD response (y-axis) and the number of measurements (x-axis) required to establish statistical significance (color-coded p-values). For a given BOLD response, the number of repeated measurements (N) required for reliable assessment (p<0.05) of a change from baseline condition lies at the right of the white dotted line. For e.g., to reliably detect a BOLD response from a voxel with peak BOLD signal response of 10%, greater than 8 measurements are needed. The bar on the right gives the scale for p values associated with the statistical significance.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Carbogen” as used herein is an admixture of carbon dioxide and oxygen. The amounts of carbon dioxide and oxygen in the admixture may be determined by one skilled in the art. Medical grade carbogen is typically 5% CO₂ and 95% O₂. In various other embodiments, carbon dioxide is used to induce hyperemia may be an admixture of ranges including but not limited to 94% O₂ and 6% CO₂, 93% O₂ and 7% CO₂, 92% O₂ and 8% CO₂, 91% O₂ and 9% CO₂, 90% O₂ and 10% CO₂, 85% O₂ and 15% CO₂, 80% O₂ and 20% CO₂, 75% O₂ and 25% CO₂ and/or 70% O₂ and 30% CO₂. Optionally, for blending with air, the CO₂ containing gas comprises 20% oxygen.

“BOLD” as used herein refers to blood-oxygen-level dependence.

A “vascular-disease-associated” coronary vasoreactive response means a type and/or quantum of vasoreactive response elicited by cardiac stress testing (e.g. exercise or administration of a hyperemic drug or a CO₂ containing gas) as demonstrable in an imaging study using one or more diagnostic imaging parameters of the type suitable to diagnose coronary vascular disease. For example, with respect to PET and SPECT, a normal response would be considered a four to five fold increase in blood flow. With respect to BOLD MRI imaging, a 10-12+% increase in BOLD signal would be considered normal. Disease associated responses are those which are not normal in varying significant degrees among which, as evidence of disease, benchmarks may be adopted to categorize differences with represent a clearer-cut diagnosis or a progression of disease that warrants greater follow-up or more proactive treatment, for example a less than two-fold increase in blood flow as measured by PET or SPECT (typically measured in ml. of blood/min/gm of tissue). Accordingly, a benchmark which represent a change from a value that clinicians described as “normal” which is at least statistically significant and optionally is also comparable to a standard for cardiac stress testing adopted by clinicians with respect to inducing stress represents a clear-cut benchmark for using CO₂ as a vasoactive stress stimulus.

A targeted increase in PaCO₂ will be selected to cause a similar vasoreactive response in normal and diseased tissue. From the standpoint of statistical significance, it will be appreciated that selection of a discriminatory increase in PaCO₂ may depend on whether or not repeat measurements are made, for example, the number of repeat measurements of a BOLD signal intensity that are made at while at lower and increased PaCO₂ levels.

Current methods for inducing hyperemia in subjects include the use of compounds such as adenosine, analogs thereof and/or functional equivalents thereof. However, such compounds (for example, adenosine) have adverse side effects including bradycardia, arrhythmia, transient or prolonged episode of asystole, ventricular fibrillation (rarely), chest pain, headache, dyspnea, and nausea, making it less than favorable for initial or follow-up studies.

The invention described herein is directed to the use of CO₂ instead of hyperemia-inducing drugs, in view of their side effects, to assess myocardial response and risk of coronary artery diseases. To date, however, it has not been possible to independently control arterial CO₂ and O₂, hence direct association of the influence of partial pressure of CO₂ (PaCO₂) on coronary vasodilation has been difficult to determine. With the development of gas flow controller devices designed to control gas concentrations in the lungs and blood (for example, RespirACT™, Thornhill Research, WO/2013/0082703), it is now possible to precisely control the arterial CO₂, while, in some embodiments, holding O₂ constant. With such devices, the desired PaCO₂ changes are rapid (1-2 breaths) and are independent of minute ventilation. The inventors are the first adopters of such devices for the assessment of myocardial response to CO₂.

The claimed invention is believed to be the first to use modulation of CO₂ levels to show that the carbon dioxide has the same effect as the clinical dose of other hyperemia-inducing drugs such as adenosine but without the side effects. The inventors induce hyperemia by administering an admixture comprising a predetermined amount of CO₂ to a subject in need thereof to assess myocardial response, evaluate coronary artery disease and identify ischemic heart disease. In an embodiment, hyperemia is induced by independently altering the administered CO₂ level while holding oxygen (O₂) constant to assess myocardial response, evaluate coronary artery disease and identify ischemic heart disease. A subject's myocardial response after administration of CO₂ may be monitored using various imaging techniques such as MRI.

Cardiac Stress Testing

When exercise stress testing is contra-indicated (in nearly 50% of patients), every existing imaging modality uses adenosine (or its analogues such as dipyridamole or regadenoson) to induce hyperemia. However, as described above, adenosine or analogs thereof or functional equivalents thereof, are well known for their adverse side effects such as bradycardia, arrhythmia, transient or prolonged episode of asystole, ventricular fibrillation (rarely), chest pain, headache, dyspnea, and nausea, making it less than favorable for initial or follow-up studies. Direct measures of ischemic burden may be determined on the basis of single-photon emission computed tomography (SPECT/SPET), positron emission tomography (PET), myocardial contrast echocardiography (MCE), and first-pass perfusion magnetic resonance imaging (FPP-MRI). SPECT and PET use radiotracers as contrast agents. While SPECT and PET studies account for approximately 90% myocardial ischemia-testing studies, the sensitivity and specificity for both methods combined for the determination of severe ischemia is below 70%. Both MCE and FPP-MRI are relatively newer approaches that require the use of exogenous contrast media and intravenous pharmacological stress agent (adenosine), both carrying significant risks and side effects in certain patient populations.

BOLD-MRI

An alternate method, BOLD (Blood-Oxygen-Level-Dependent) MRI, relies on endogenous contrast mechanisms (changes in blood oxygen saturation, % O₂) to identify ischemic territories. The potential benefits of BOLD MRI for detecting global or regional myocardial ischemia due to coronary artery disease (CAD) were demonstrated by the inventors and others at least a decade ago. Although a number of pilot clinical studies have demonstrated the feasibility of using BOLD MRI for identifying clinically significant myocardial ischemia due to CAD, the method is inherently limited by sensitivity and specificity due to low BOLD contrast-to-noise ratio (CNR). The repeatability of BOLD MRI using CO₂ provides the means to improve sensitivity and specificity, which is not possible using adenosine or analogs thereof.

The invention provides a method for increasing the sensitivity and specificity of BOLD MRI. The method includes administering an admixture comprising of CO₂ to the subject in need thereof to induce hyperemia and imaging the myocardium using MRI to assess a hypermic response in response to a predetermined modulation in PaCO₂.

The proposed method utilizes (i) an individualized targeted change in arterial partial pressure of CO₂ (PaCO₂) as the non-invasive vasoactive stimulus, (ii) fast, high-resolution, 4D BOLD MRI at 3T and (iii) statistical models (for example, the generalized linear model (GLM) theory) to derive statistical parametric maps (SPM) to reliably detect and quantify the prognostically significant ischemic burden through repeated measurements (i.e. in a data-driven fashion).

The method for increasing the sensitivity and specificity of BOLD MRI comprises (i) obtaining free-breathing cardiac phase-resolved 3D myocardial BOLD images (under different PaCO₂ states established via inhalation of an admixture of gases comprising of CO₂); (ii) registering and segmenting the images to obtain the myocardial dynamic volume and (iii) identifying ischemic territory and quantify image volume.

Obtaining the Images

The first step in increasing the sensitivity and specificity of BOLD MRI is to obtain free-breathing cardiac phase resolved 3D myocardial BOLD images. Subjects are placed on the MRI scanner table, ECG leads are placed, and necessary surface coils are positioned. Subsequently their hearts are localized and the cardiac shim protocol is prescribed over the whole heart. K-space lines, time stamped for trigger time are collected using cine SSFP acquisition with image acceleration along the long axis. Central k-space lines corresponding to each cardiac phase will be used to derive the center of mass (COM) curves along the z-axis via 1-D fast Fourier transform (FFT). Based on the COM curves, the k-space lines from each cardiac phase will be sorted into 1-30 bins, each corresponding to a respiratory state with the first bin being the reference bin (end-expiration) and the last bin corresponding to end inspiration.

To minimize the artifacts from under sampling, the data will be processed with a 3D filter, followed by re-gridding the k-space lines, application of a spatial mask (to restrict the registration to region of the heart) and performing FFT to obtain the under sampled 3D image for each respiratory bin. Using the end-expiration image as the reference image, images from all bins (except bin 1) are registered using kits such as Insight Tool Kit (freely available from www.itk.org), or an equivalent software platform, in an iterative fashion and the transform parameters will be estimated for rotation, scaling, shearing, and translation of heart between the different respiratory bins. The k-space data will again be divided into 1 to 30 respiratory bins, re-gridded, transformed to the reference image (3D affine transform), summed together, and the final 3D image will be reconstructed. Imaging parameters may be TR=3.0 to 10 ms and flip angle=1° to 90°. In this fashion, 3D cine data under controlled PaCO₂ values (hypo- and hyper-carbic states) are collected.

Registration and Segmentation of Images

The next step in increasing the sensitivity and specificity of BOLD MRI is registration and segmentation of the images to obtain the myocardial dynamic volume. The pipeline utilizes MATLAB and C++ using the ITK framework or an equivalent software platform. The myocardial MR images obtained with repeat CO₂ stimulation blocks will be loaded in MATLAB (or an equivalent image processing platform) and arranged in a four-dimensional (4D) matrix, where the first 3 dimensions represent volume (voxels) and the fourth dimension is time (cardiac phase). Subsequently, each volume is resampled to achieve isotropic voxel size. End-systole (ES) are identified for each stack based on our minimum cross-correlation approach. A 4D non-linear registration algorithm is used to find voxel-to-voxel correspondences (deformation fields) across all cardiac phases. Using the recovered deformation, all cardiac phases are wrapped to the space of ES, such that all phases are aligned to ES. Recover the transformations across all ES images from repeat CO₂ blocks and bring them to the same space using a diffeomorphic volume registration tool, such as ANTs. Upon completion, all cardiac phases from all acquisitions will be spatially aligned to the space of ES of the first acquisition (used as reference) and all phase-to-phase deformations and acquisition-to-acquisition transformations will be known. An expert delineation of the myocardium in the ES of the first (reference) acquisition will then be performed. Based on the estimated deformation fields and transformations, this segmentation is propagated to all phases and acquisitions, resulting in fully registered and segmented myocardial dynamic volumes.

Image Analysis to Identity and Quantify Ischemic Territories

The final step needed for increasing the sensitivity and specificity of BOLD MRI is identifying ischemic territory and quantify image volume. Since BOLD responses are optimally observed in systolic frames, only L systolic cardiac volumes (centered at ES) are retained from each fully registered and segmented 4D BOLD MR image set obtained above. Only those voxels contained in the myocardium are retained and the corresponding RPP (rate-pressure-product) and PaCO₂ are noted. Assuming N acquisitions per CO₂ state (hypocarbic or hypercarbic) and K, CO₂ stimulation blocks, and each cardiac volume consists of n×m×p (×=multiplication) isotropic voxels, build a concatenated fully registered 4D dataset consisting of n×m×p×t pixels, where ×=multiplication and t=L×K×N, and export this dataset in NIFTI (or an equivalent) format using standard tools. The 4D dataset is loaded into a voxel-based statistical model fitting (such as FSL-FEAT developed for fMRI), to fit the model for each voxel. The statistical analysis outputs a P-statistic volume, i.e., the SPM, where for each voxel in the myocardium the p-value of the significance of the correlation to the model is reported. The statistical parametric maps (SPM) are thresholded by identifying the voxels that have p<0.05. Those voxels are identified as hyperemic for responding to the CO₂ stimulation. The total number of hyperemic voxels (V_{H}) are counted and their relative volume (V_{RH}=V_{H}/total voxels in myocardium) is determined. The voxels that do not respond to CO₂ stimulation (on SPM) are identified as ischemic and used to generate a binary 3D map of ischemic voxels (3D-ISCHₘₐₚ). In addition, total ischemic voxels (V_{I}) and the relative ischemic volume (V_{RI}=V_{I}/total myocardial voxels) are determined.

The above methods provide ischemic volumes that can be reliably identified on the basis of statistical analysis applied to repeatedly acquire 4D BOLD images under precisely targeted changes in PaCO₂. These volumes are closely related to the clinical index of fractional flow reserve FFR.

FFR

An additional method, fractional flow reserve (FFR) is used in coronary catheterization to measure pressure differences across a coronary artery stenosis to determine the likelihood that the stenosis impedes oxygen delivery to the heart muscle (myocardial ischemia). Fractional flow reserve measures the pressure behind (distal to) a stenosis relative to the pressure before the stenosis, using adenosine or papaverine to induce hyperemia. A cut-off point of 0.75 to 0.80 has been used wherein higher values indicate a non-significant stenosis and lower values indicate a significant lesion. FFR, determined as the relative pressure differences across the stenotic coronary artery has emerged as the new standard for determining clinically significant ischemia (FFR≤0.75). However, it is invasive, expensive, and exposes the patient to ionizing radiation and the side-effects of the use of adenosine. In view of the side-effects of adenosine discussed above, Applicants propose using carbon dioxide instead of adenosine to induce hyperemia, by administering to a subject an admixture comprising CO₂ to reach a predetermined PaCO₂ in the subject to induce hyperemia. In some embodiments, the admixture comprises any one or more of carbon dioxide, oxygen and nitrogen; carbon dioxide and oxygen; carbon dioxide and nitrogen; or carbon dioxide alone. In one embodiment, the amounts of CO₂ and O₂ administered are both altered. In another embodiment, the amount of CO₂ administered is altered to a predetermined level while the amount of O₂ administered is held constant. In various embodiments, the amounts of any one or more of CO₂, O₂ or N₂ in an admixture are changed or held constant as would be readily apparent to a person having ordinary skill in the art.

Methods of the Invention

The invention is directed to methods for diagnosing coronary heart disease in a subject in need thereof comprising administering an admixture comprising CO₂ to a subject to reach a predetermined PaCO₂ in the subject to induce hyperemia, monitoring vascular reactivity in the subject and diagnosing the presence or absence of coronary heart disease in the subject, wherein decreased vascular reactivity in the subject compared to a control subject is indicative of coronary heart disease. In an embodiment, CO₂ is administered via inhalation. In another embodiment, CO₂ levels are altered while the O₂ levels remain unchanged so that the PaCO₂ is changed independently of the O₂ level. In a further embodiment, vascular reactivity is monitored using imagining techniques deemed appropriate by one skilled in the art, including but not limited to any one or more of positron emission tomography (PET), single photon emission computed tomography/computed tomography (SPECT), computed tomography (CT), magnetic resonance imaging (MRI), functional magnetic resonance imaging (fMRI), single photon emission computed tomography/computed tomography (SPECT/CT), positron emission tomography/computed tomography (PET/CT), ultrasound, electrocardiogram (ECG), Electron-beam computed tomography (EBCT), echocardiogram (ECHO), electron spin resonance (ESR) and/or any combination of the imaging modalities such as (PET/MR), PET/CT, and/or SPECT/MR. In an embodiment, vascular reactivity is monitored using free-breathing BOLD MRI. In some embodiments, the admixture comprises any one or more of carbon dioxide, oxygen and nitrogen; carbon dioxide and oxygen; carbon dioxide and nitrogen; or carbon dioxide alone. In one embodiment, the amounts of CO₂ and O₂ administered are both altered. In another embodiment, the amount of CO₂ administered is altered to a predetermined level while the amount of O₂ administered is held constant. In various embodiments, the amounts of any one or more of CO₂, O₂ or N₂ in an admixture are changed or held constant as would be readily apparent to a person having ordinary skill in the art.

The invention also provides a method for assessing hyperemic response in a subject in need thereof comprising administering an admixture comprising CO₂ to a subject to reach a predetermined PaCO₂ in the subject to induce hyperemia, monitoring vascular reactivity in the subject and assessing hyperemic response in the subject, wherein decreased vascular reactivity in the subject compared to a control subject is indicative of poor hyperemic response, thereby assessing hyperemic response in the subject in need thereof. This method may also be used to assess organ perfusion and assess vascular reactivity. In an embodiment, CO₂ is administered via inhalation. In another embodiment, CO₂ levels are altered while the O₂ levels remain unchanged so that the PaCO₂ is changed independently of the O₂ level. In a further embodiment, vascular reactivity is monitored using imagining techniques deemed appropriate by one skilled in the art, including but not limited to any one or more of positron emission tomography (PET), single photon emission computed tomography/computed tomography (SPECT), computed tomography (CT), magnetic resonance imaging (MRI), functional magnetic resonance imaging (fMRI), single photon emission computed tomography/computed tomography (SPECT/CT), positron emission tomography/computed tomography (PET/CT), ultrasound, electrocardiogram (ECG), Electron-beam computed tomography (EBCT), echocardiogram (ECHO), electron spin resonance (ESR) and/or any combination of the imaging modalities such as (PET/MR), PET/CT, and/or SPECT/MR. In an embodiment, vascular reactivity is monitored using free-breathing BOLD MRI. In some embodiments, the admixture comprises any one or more of carbon dioxide, oxygen and nitrogen; carbon dioxide and oxygen; carbon dioxide and nitrogen; or carbon dioxide alone. In one embodiment, the amounts of CO₂ and O₂ administered are both altered. In another embodiment, the amount of CO₂ administered is altered to a predetermined level while the amount of O₂ administered is held constant. In various embodiments, the amounts of any one or more of CO₂, O₂ or N₂ in an admixture are changed or held constant as would be readily apparent to a person having ordinary skill in the art.

The invention is further directed to methods for producing coronary vasodilation in a subject in need thereof comprising providing a composition comprising CO₂ and administering the composition comprising CO₂ to a subject to reach a predetermined PaCO₂ in the subject so as to produce coronary vasodilation in the subject, thereby producing coronary vasodilation in the subject. In an embodiment, CO₂ is administered via inhalation. In another embodiment, CO₂ levels are altered while the O₂ levels remain unchanged so that the PaCO₂ is changed independently of the O₂ level. In a further embodiment, vascular reactivity is monitored using imagining techniques deemed appropriate by one skilled in the art, including but not limited to any one or more of positron emission tomography (PET), single photon emission computed tomography/computed tomography (SPECT), computed tomography (CT), magnetic resonance imaging (MRI), functional magnetic resonance imaging (fMRI), single photon emission computed tomography/computed tomography (SPECT/CT), positron emission tomography/computed tomography (PET/CT), ultrasound, electrocardiogram (ECG), Electron-beam computed tomography (EBCT), echocardiogram (ECHO), electron spin resonance (ESR) and/or any combination of the imaging modalities such as (PET/MR), PET/CT, and/or SPECT/MR. In an embodiment, vascular reactivity is monitored using free-breathing BOLD MRI. In some embodiments, the admixture comprises any one or more of carbon dioxide, oxygen and nitrogen; carbon dioxide and oxygen; carbon dioxide and nitrogen; or carbon dioxide alone. In one embodiment, the amounts of CO₂ and O₂ administered are both altered. In another embodiment, the amount of CO₂ administered is altered to a predetermined level while the amount of O₂ administered is held constant. In various embodiments, the amounts of any one or more of CO₂, O₂ or N₂ in an admixture are changed or held constant as would be readily apparent to a person having ordinary skill in the art.

The invention also provides a method for assessing tissue and/or organ perfusion in a subject in need thereof comprising administering an admixture comprising CO₂ to a subject to reach a predetermined PaCO₂ in the subject to induce hyperemia, monitoring vascular reactivity in the tissue and/or organ and assessing tissue and/or organ perfusion by assessing the hyperemic response in the subject, wherein decreased vascular reactivity in the subject compared to a control subject is indicative of poor hyperemic response and therefore poor tissue and/or organ perfusion. In an embodiment, CO₂ is administered via inhalation. In another embodiment, CO₂ levels are altered while the O₂ levels remain unchanged so that the PaCO₂ is changed independently of the O₂ level. In a further embodiment, vascular reactivity is monitored using imagining techniques deemed appropriate by one skilled in the art, including but not limited to any one or more of positron emission tomography (PET), single photon emission computed tomography/computed tomography (SPECT), computed tomography (CT), magnetic resonance imaging (MRI), functional magnetic resonance imaging (fMRI), single photon emission computed tomography/computed tomography (SPECT/CT), positron emission tomography/computed tomography (PET/CT), ultrasound, electrocardiogram (ECG), Electron-beam computed tomography (EBCT), echocardiogram (ECHO), electron spin resonance (ESR) and/or any combination of the imaging modalities such as (PET/MR), PET/CT, and/or SPECT/MR. In an embodiment, vascular reactivity is monitored using free-breathing BOLD MRI. In some embodiments, the admixture comprises any one or more of carbon dioxide, oxygen and nitrogen; carbon dioxide and oxygen; carbon dioxide and nitrogen; or carbon dioxide alone. In one embodiment, the amounts of CO₂ and O₂ administered are both altered. In another embodiment, the amount of CO₂ administered is altered to a predetermined level while the amount of O₂ administered is held constant. In various embodiments, the amounts of any one or more of CO₂, O₂ or N₂ in an admixture are changed or held constant as would be readily apparent to a person having ordinary skill in the art.

In some embodiments, the admixture comprising CO₂ is administered at high doses for short duration or at low doses for longer durations. For example, administering the admixture comprising CO₂ at high doses of CO₂ for a short duration comprises administering any one or more of 40 mmHg to 45 mmHg, 45 mmHg to 50 mmHg, 50 mmHg to 55 mmHg, 55 mmHg CO₂ to 60 mm Hg CO₂, 60 mmHg CO₂ to 65 mm Hg CO₂, 65 mmHg CO₂ to 70 mm Hg CO₂, 70 mmHg CO₂ to 75 mm Hg CO₂, 75 mmHg CO₂ to 80 mm Hg CO₂, 80 mmHg CO₂ to 85 mm Hg CO₂ or a combination thereof, for about 20 minutes, 15 minutes, 10 minutes, 9 minutes, 8 minutes, 7 minutes, 6 minutes, 5 minutes, 4 minutes, 3 minutes, 2 minutes, 1 minute or a combination thereof. In various embodiments, the predetermined levels of CO₂ are administered so that the arterial level of CO₂ reaches the PaCO₂ of any one or more of the above ranges.

For example, administering low doses of predetermined amounts of CO₂ for a longer duration comprises administering the predetermined amount of CO₂ at any one or more of about 30 mmHg CO₂ to about 35 mmHg CO₂, about 35 mmHg CO₂ to about 40 mmHg CO₂, about 40 mmHg CO₂ to about 45 mmHg CO₂or a combination thereof for any one or more of about 20 to 24 hours, about 15 to 20 hours, about 10 to 15 hours, about 5 to 10 hours, about 4 to 5 hours, about 3 to 4 hours, about 2 to 3 hours, about 1 to 2 hours, or a combination thereof, before inducing hyperemia. In various embodiments, the predetermined levels of CO₂ are administered so that the arterial level of CO₂ reaches the PaCO₂ of any one or more of the above ranges.

In one embodiment, CO₂ is administered in a stepwise manner. In another embodiment, administering carbon dioxide in a stepwise manner includes administering carbon dioxide in 5 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

In another embodiment, administering carbon dioxide in a stepwise manner includes administering carbon dioxide in 10 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

In a further embodiment, administering carbon dioxide in a stepwise manner includes administering carbon dioxide in 20 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

In a further embodiment, administering carbon dioxide in a stepwise manner includes administering carbon dioxide in 30 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

In a further embodiment, administering carbon dioxide in a stepwise manner includes administering carbon dioxide in 40 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

In a further embodiment, administering carbon dioxide in a stepwise manner includes administering carbon dioxide in 50 mmHg increments in the range of any one or more of 10 mmHg to 100 mmHg CO₂, 20 mmHg to 100 mmHg CO₂, 30 mmHg to 100 mmHg CO₂, 40 mmHg to 100 mmHg CO₂, 50 mmHg to 100 mmHg CO₂, 60 mmHg to 100 mmHg CO₂, 10 mmHg to 90 mmHg CO₂, 20 mmHg to 90 mmHg CO₂, 30 mmHg to 90 mmHg CO₂, 40 mmHg to 90 mmHg CO₂, 50 mmHg to 90 mmHg CO₂, 60 mmHg to 90 mmHg CO₂, 10 mmHg to 80 mmHg CO₂, 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 10 mmHg to 70 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 10 mmHg to 60 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂. In various embodiments, the predetermined levels of CO₂ are administered so that the arterial level of CO₂ reaches the PaCO₂ of any one or more of the above ranges.

Other increments of carbon dioxide to be administered in a stepwise manner will a readily apparent to a person having ordinary skill in the art.

In a further embodiment, predetermined amount of CO₂ is administered in a block manner. Block administration of carbon dioxide comprises administering carbon dioxide in alternating amounts over a period of time. In alternating amounts of CO₂ comprises alternating between any of 20 mmHg and 40 mmHg, 30 mmHg and 40 mmHg, 20 mmHg and 50 mmHg, 30 mmHg and 50 mmHg, 40 mmHg and 50 mmHg, 20 mmHg and 60 mmHg, 30 mmHg and 60 mmHg, 40 mmHg and 60 mmHg, or 50 mmHg and 60 mmHg. In various embodiments, the predetermined levels of CO₂ are administered so that the arterial level of CO₂ reaches the PaCO₂ of any one or more of the above ranges. Other amounts of carbon dioxide to be used in alternating amounts over a period of time will be readily apparent to a person having ordinary skill in the art.

In one embodiment, vascular reactivity may be measured by characterization of Myocardial Perfusion Reserve, which is defined as a ratio of Myocardial Perfusion at Stress to Myocardial Perfusion at Rest. In healthy subjects the ratio may vary from 5:1 to 6:1. The ratio diminishes with disease. A decrease in this ratio to 2:1 from the healthy level is considered the clinically significant and indicative of poor vascular reactivity.

In another embodiment, vascular reactivity may be measured via differential absolute perfusion, which may be obtained using imaging methods such as first pass perfusion, SPECT/PET, CT perfusion or echocardiography in units of ml/sec/g of tissue.

In an embodiment the CO₂ gas is administered via inhalation. CO₂ may be administered using, for example, RespirACT™ technology from Thornhill Research. In various embodiments, CO₂ is administered for 1-2 minutes, 2-4 minutes, 4-6 minutes, 6-8 minutes, 8-10 minutes, 10-12 minutes, 12-14 minutes, 14-16 minutes, 16-18 minutes and/or 18-20 minutes. In a preferred embodiment, CO₂ is administered for 4-6 minutes. In an additional embodiment CO₂ is administered for an amount of time it takes for the arterial PaCO₂ (partial pressure of carbon dioxide) to reach 50-60 mmHg from the standard levels of 30 mmHg during CO₂-enhanced imaging.

In one embodiment, carbon dioxide used to induce hyperemia is medical-grade carbogen which is an admixture of 95% O₂ and 5% CO₂. In various other embodiments, carbon dioxide is used to induce hyperemia may be an admixture of ranges including but not limited to 94% O₂ and 6% CO₂, 93% O₂ and 7% CO₂, 92% O₂ and 8% CO₂, 91% O₂ and 9% CO₂, 90% O₂ and 10% CO₂, 85% O₂ and 15% CO₂, 80% O₂ and 20% CO₂, 75% O₂ and 25% CO₂ and/or 70% O₂ and 30% CO₂.

In another embodiment, vascular reactivity and/or vasodilation are monitored using any one or more of positron emission tomography (PET), single photon emission computed tomography/computed tomography (SPECT), computed tomography (CT), magnetic resonance imaging (MRI), functional magnetic resonance imaging (fMRI), single photon emission computed tomography/computed tomography (SPECT/CT), positron emission tomography/computed tomography (PET/CT), ultrasound, electrocardiogram (ECG), Electron-beam computed tomography (EBCT), echocardiogram (ECHO), electron spin resonance (ESR) and/or any combination of the imaging modalities such as (PET/MR), PET/CT, and/or SPECT/MR In an embodiment, vascular reactivity is monitored using free-breathing BOLD MRI.

Imaging techniques using carbon dioxide involve a free-breathing approach so as to permit entry of CO₂ into the subject's system. In an embodiment, the subjects include mammalian subjects, including, human, monkey, ape, dog, cat, cow, horse, goat, pig, rabbit, mouse and rat. In a preferred embodiment, the subject is human.

### ADVANTAGES OF THE INVENTION

The methods described herein to functionally assess the oxygen status of the myocardium include administering an effective amount of CO₂ to the subject in need thereof In an embodiment, the O₂ level is held constant while the CO₂ level is altered so as to induce hyperemia. Applicants herein show the vascular reactivity in subjects in response to changes in PaCO₂. The existing methods use adenosine, dipyridamole, or regadenoson which have significant side-effects described above. As described in the Examples below, CO₂ is at least just as effective as the existing methods (which use, for example, adenosine) but without the side effects.

The use of CO₂ provides distinct advantages over the use of, for example, adenosine. Administering CO₂ is truly non-invasive because it merely involves inhaling physiologically sound levels of CO₂. The instant methods are simple, repeatable and fast and most likely have the best chance for reproducibility. Not even breath-holding is necessary during acquisition of images using the methods described herein. The instant methods are also highly cost-effective as no pharmacological stress agents are required, cardiologists may not need to be present during imaging and rapid imaging reduces scan times and costs.

Further, the improved BOLD MRI technique described above provides a non-invasive and reliable determination of ischemic volume (no radiation, contrast-media, or adenosine) and other value-added imaging biomarkers from the same acquisition (Ejection Fraction, Wall Thickening). Additionally, the subject does not experience adenosine-related adverse side effects and thus greater patient tolerance for repeat ischemia testing. There is a significant cost-savings from abandoning exogenous contrast media and adenosine/regadenoson. Moreover, the proposed BOLD MRI paradigm will be accompanied by significant technical advances as well: (i) a fast, high-resolution, free-breathing 4D SSFP MRI at 3T, that can impact cardiac MRI in general; (ii) Repeated stimulations of the heart via precisely targeted changes in PaCO₂; and (iii) adoption of sophisticated analytical methods employed in the brain to the heart.

### EXAMPLES

All imaging studies were performed in instrumented animals with a Doppler flow probe attached to the LAD coronary arteries for measurement of flow changes in response to CO₂ and clinical dose of adenosine. In these studies, CO₂ and O₂ delivery were tightly controlled using Respiract. CO₂ values were incremented in steps of 10 mmHg starting from 30 mmHg to 60 mmHg and were ramped down in decrements of 10 mmHg. At each CO₂ level, free-breathing and cardiac gated blood-oxygen-level-dependent (BOLD) acquisitions were prescribed at mid diastole and Doppler flow velocities were measured. Similar acquisitions were also performed with block sequences of CO₂ levels; that is, CO₂ levels were alternated between 40 and 50 mmHg and BOLD images (and corresponding Doppler flow velocities) were acquired at each CO₂ level to assess the reproducibility of the signal changes associated with different CO₂ levels. Each delivery of CO₂ using Respiract were made in conjunction with arterial blood draw to determine the arterial blood CO₂ levels. Imaging-based demonstration of myocardial hyperemic response to changes in PaCO₂ was shown in health human volunteers with informed consent.

### Example 1

The inventor has shown that CO₂ can increase myocardial perfusion by a similar amount, as does adenosine in canine models. The inventor has also shown that in the setting of coronary artery narrowing, it is possible to detect regional variations in hyperemic response with the use of MRI by detecting signal changes in the myocardium due to changes in oxygen saturation (also known as the BOLD effect) using a free-breathing BOLD MRI approach.

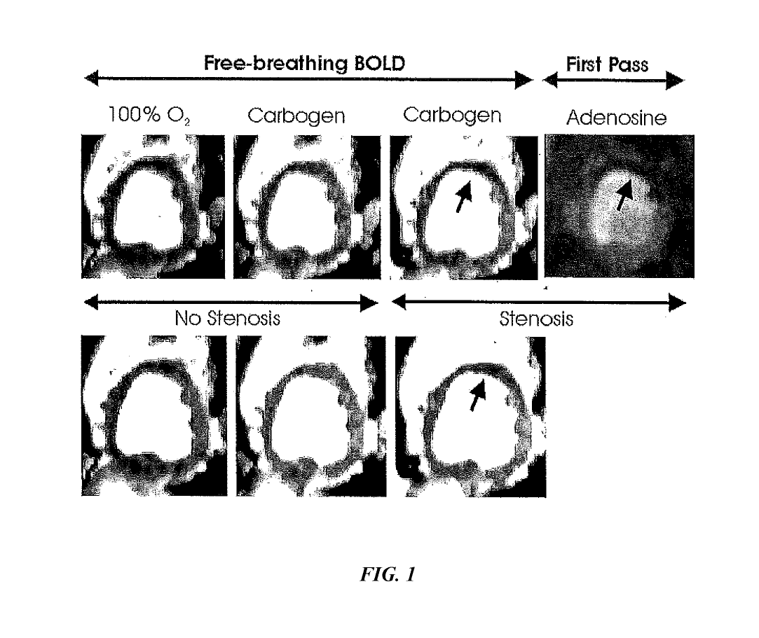

As show in FIG. 1, the inventor found a 20% BOLD signal increase (hyperemic response) with medical-grade carbogen breathing in the absence of stenosis in dogs. With a severe stenosis, the hyperemic response was significantly reduced in the LAD (left anterior descending) territory but the other regions showed an increase in signal intensity (as observed with adenosine). First-pass perfusion images acquired with adenosine under severe stenosis (in the same slice position and trigger time) is also shown for comparison. Heart rate increase of around 5-10% and a drop in blood pressure (measured invasively) by about 5% was also observed in this animal under carbogen. All acquisitions were navigator gated T2-prep 2D SSFP (steady-state free precession) and triggered at mid/end diastole (acquisition window of 50 ms). To date 10 dogs have been studied with medical-grade carbogen and have yielded highly reproducible results.

In detail, the color images (lower panel of FIG. 1) are color-coded to the signal intensities of grayscale images (above). The darker colors (blue/black) represent territories of baseline myocardial oxygenation and the brighter regions represent those regions that are hyperemic. On average the signal difference between a dark blue (low signal) and orange color (high signal) is about 20%. Note that in the absence of stenosis, as one goes from 100% O₂ to Carbogen, the BOLD signal intensity is elevated (second image from left) suggesting CO₂ based vasoreactivity of the myocardium. The dogs were instrumented with an occluder over the left-anterior descending (LAD) coronary artery. As the LAD is occluded, note that the region indicated by an arrow (i.e. approximately between 11 o'clock and 1-2 o'clock (region supplied by the LAD)) becomes darker (3rd image from left), suggesting that vasodilation is no longer possible or is reduced. The first pass image (obtained with adenosine stress following BOLD images) at the same stenosis level also shows this territory clearly. The inventor has also been comparing the epicardial flow enhancements in response to Carbogen (with ETCO2 reaching 48-50 mm Hg) against clinical dose of adenosine and the responses have been quite similar (˜20% response).

### Example 2

Co-Relation Between Inhaled CO₂ and Oxygen Saturation

Applicants assessed the difference between myocardial blood-oxygen-level dependent (BOLD) response under hypercarbia and normocarbia conditions in canines. The BOLD signal intensity is proportional to oxygen saturation.

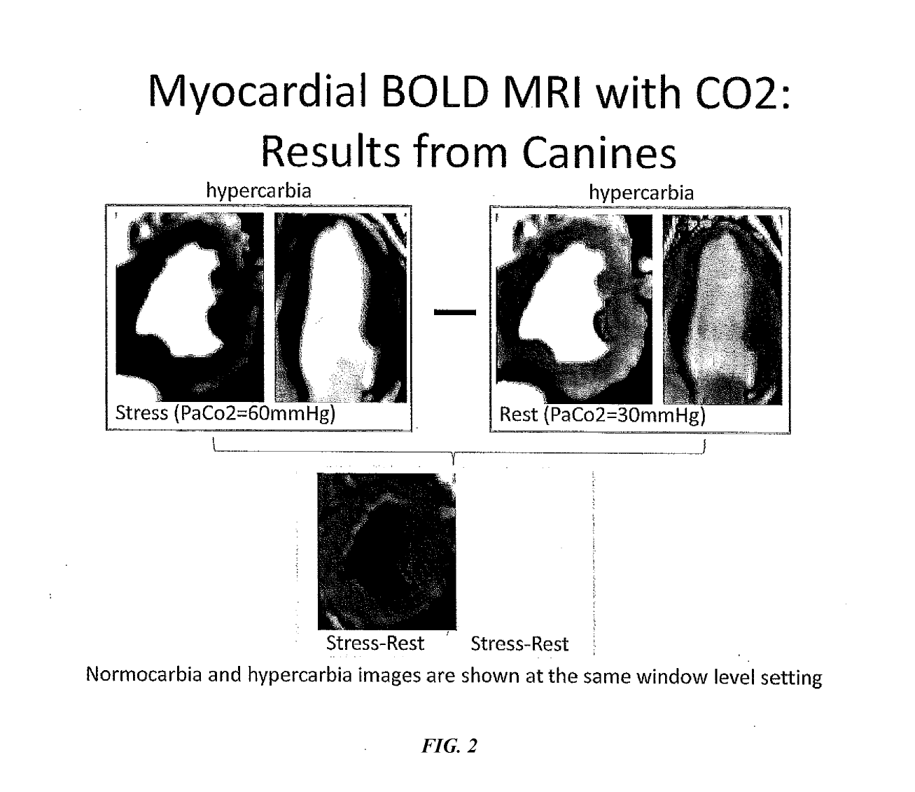

Top panels of FIG. 2 depict the myocardial response under hypercarbia (60 mm Hg) and normocarbia (30 mmHg) conditions and show an increase in BOLD signal intensity under hypercarbia condition. The lower panel depicts the difference as obtained by subtracting the signal under rest from that under stress. The myocardial BOLD signal difference between the two is depicted in grey and shows the responsiveness of canines to hypercarbia conditions.

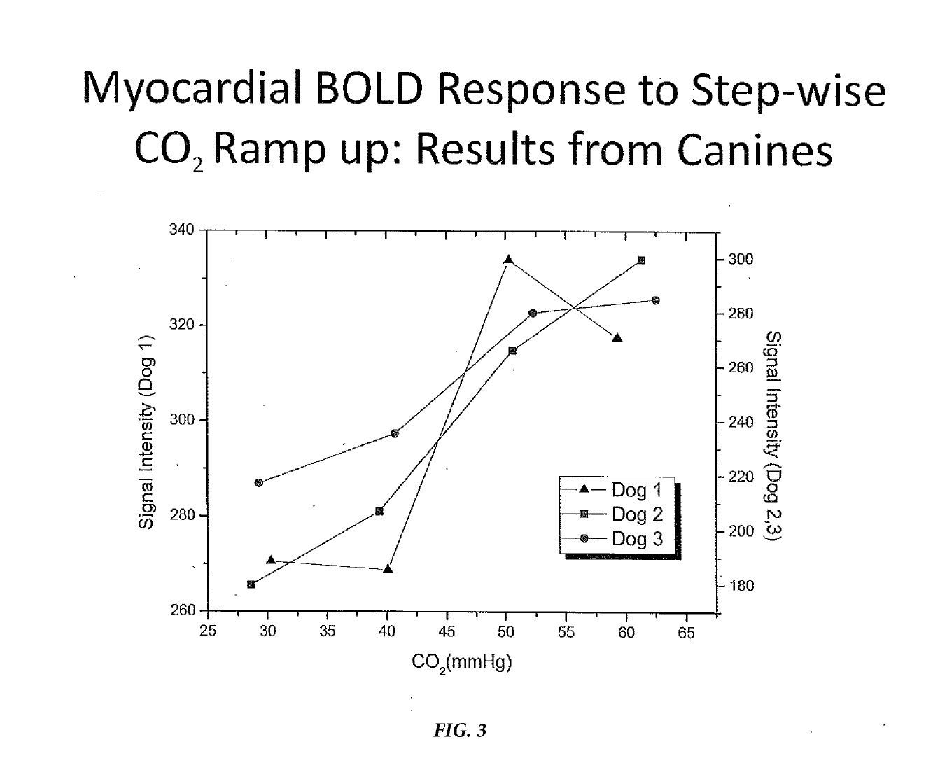

Applicants further assessed the myocardial BOLD response to stepwise CO₂ increase (ramp-up) in canines. As shown in FIG. 3, as the amount of CO₂ administered increases, the BOLD signal intensity increases which is indicative of an increase in hyperemic response to increased uptake of CO₂ and oxygen saturation.

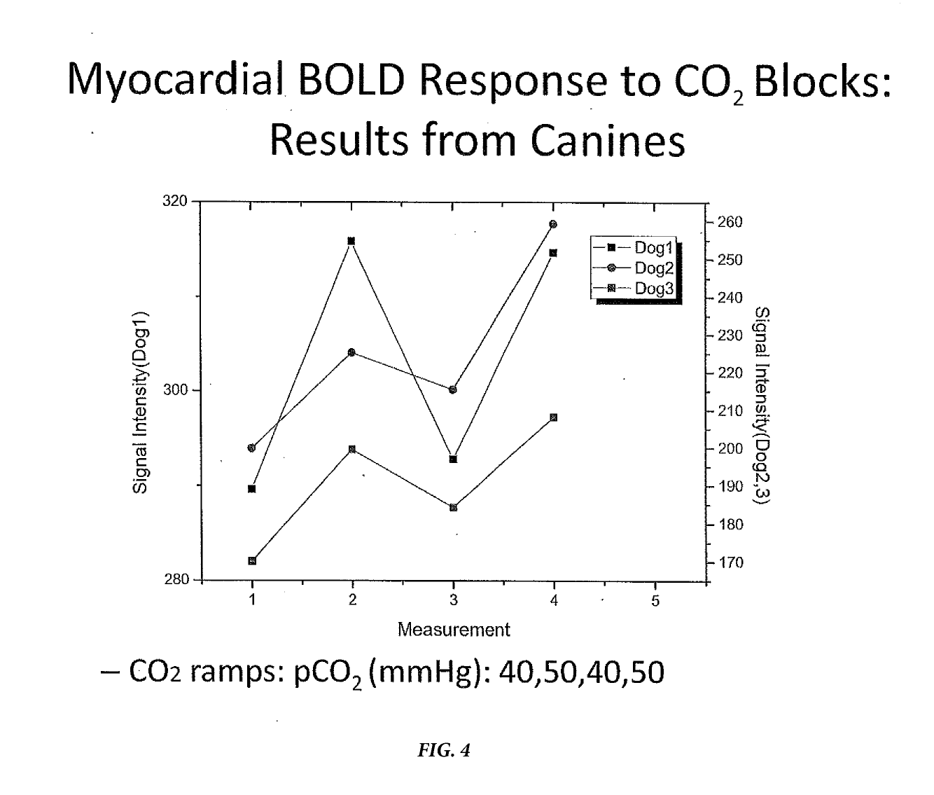

To further evaluate vascular reactivity and coronary response to CO₂, Applicants measured the myocardial BOLD signal in response to block increases of CO₂ in canines. Specifically, the myocardial BOLD signal was measured as the amount of CO₂ administered to the canine subjects alternated between 40 mmHg CO₂ and 50 mmHg CO₂. As shown in FIG. 4, an increase in CO₂ level from 40 mmHg CO₂ to 50 mmHg CO₂ resulted in an increase in BOLD signal intensity and the subsequent decrease in CO₂ level to 40 mmHg resulted in a decreased BOLD signal. These results show a tight co-relation between administration of CO₂ and vascular reactivity and coronary response.

### Example 3

Co-Relation Between the Amount of CO₂ Inhaled and Doppler Flow

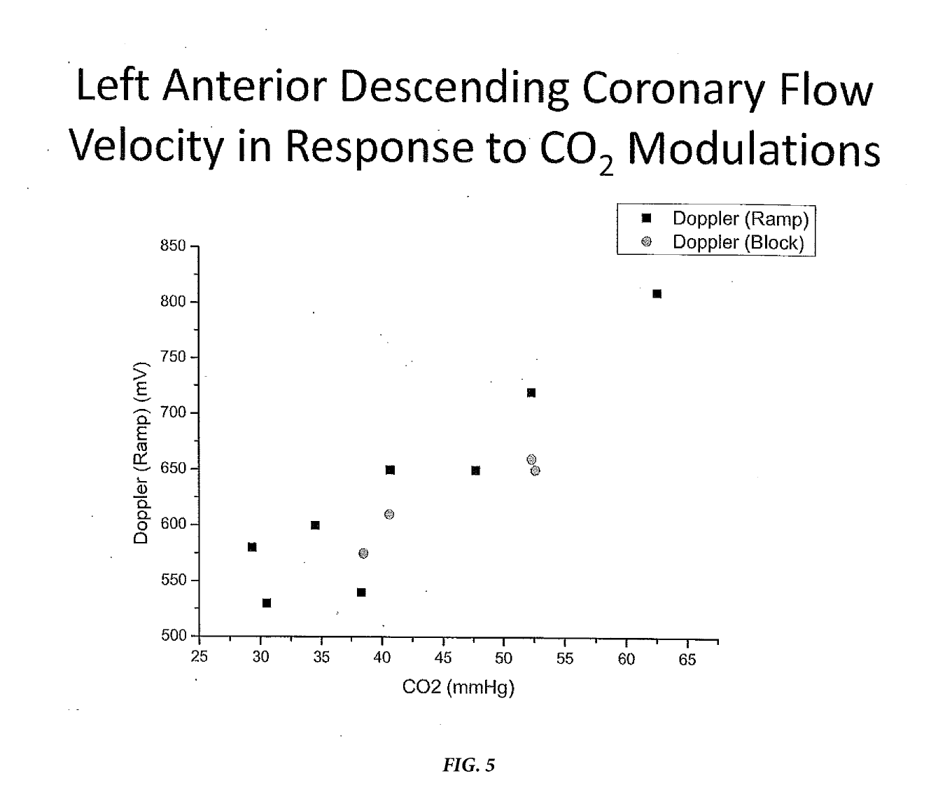

Doppler flow, an ultrasound-based approach which uses sound waves to measure blood flow, was used to show that administration of CO₂ leads to vasodilation which results in greater blood flow, while PaO₂ is held constant. The Doppler flow was measured in the left anterior descending (LAD) artery. As shown in FIG. 5, as the amount of administered CO₂ increases the Doppler flow increases. The relative change in coronary flow velocity is directly proportional to the amount of CO₂ administered.

### Example 4

Each of the Arteries which Supply Blood to the Myocardium Responds to the CO₂ Levels

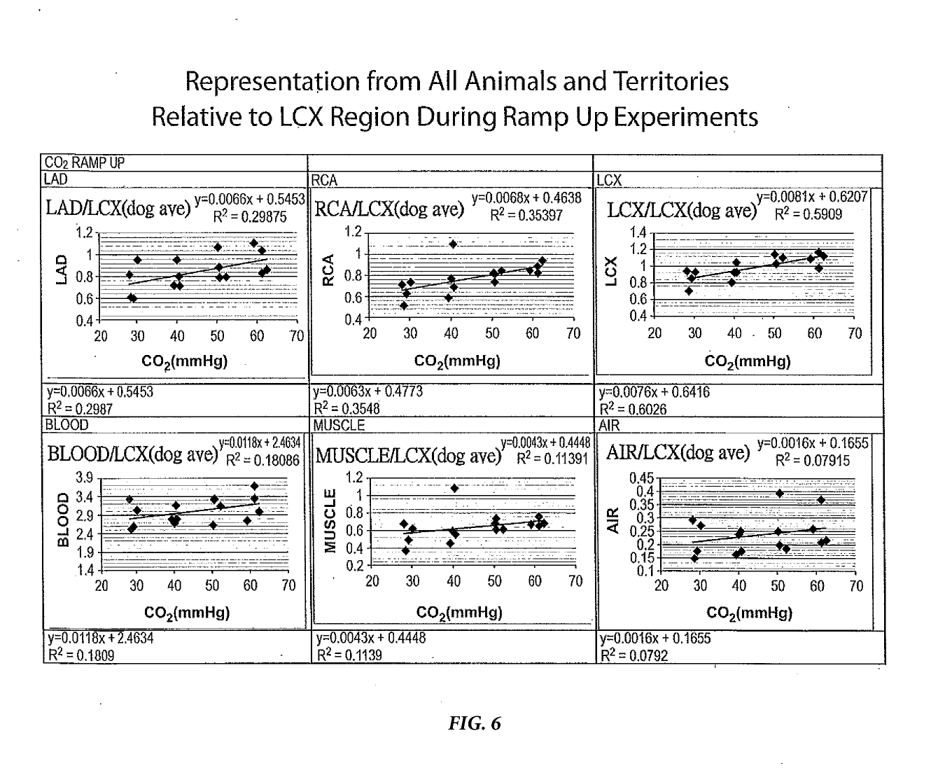

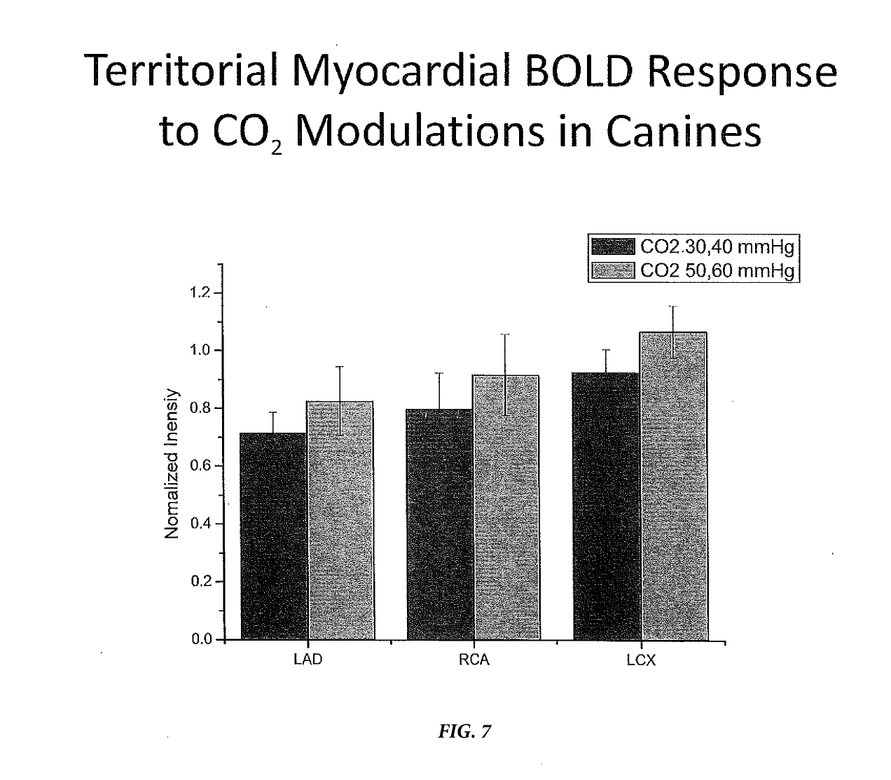

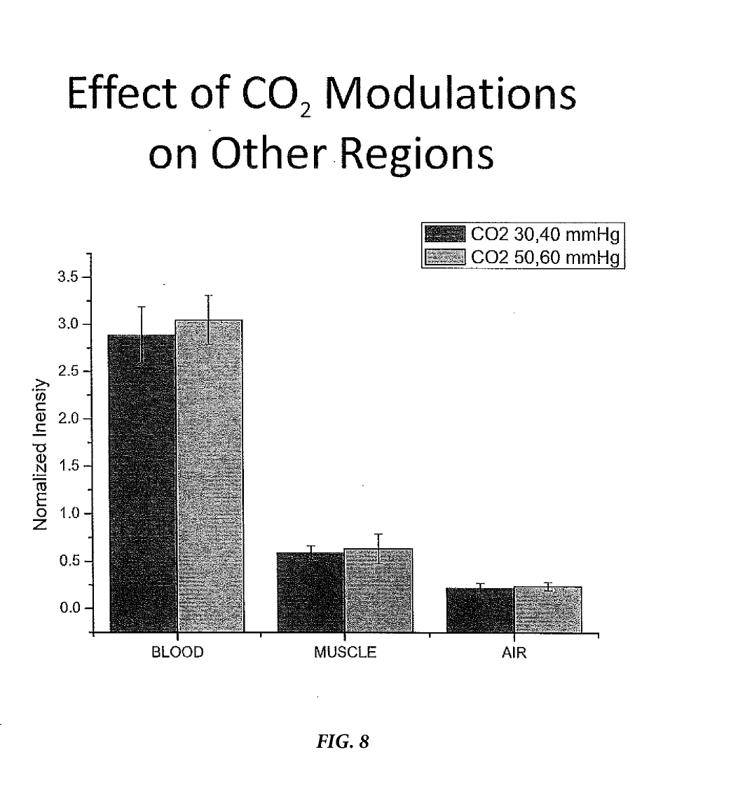

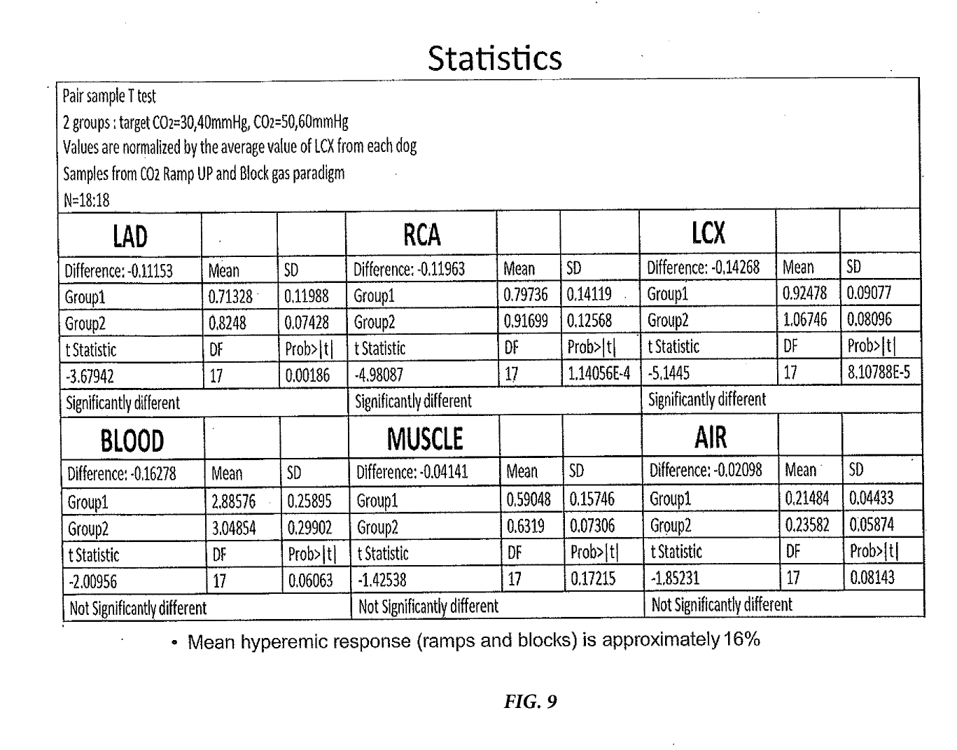

The myocardium is supplied with blood by the left anterior descending (LAD) artery, the right coronary artery (RCA) and the left circumflex (LCX) artery. Applicants measured the blood flow through each of these arteries in response to increasing CO2 supply. As shown in FIG. 6 and summarized in FIG. 7, in each of the three LAD, RCA and LCX arteries, there is a direct correlation between the amount of CO₂ administered and the signal intensity; as the amount of administered CO₂ increases, the signal intensity, signaling the blood flow, in each of the three arteries increases. Further, as shown in FIG. 6 and summarized in FIG. 8, there is no response to CO₂ modulation in control territories such as blood, skeletal muscle or air. As shown in FIG. 9, the mean hyperemic response is approximately 16%.

### Example 5

Vascular Reactivity to CO₂ Comparable to Adenosine

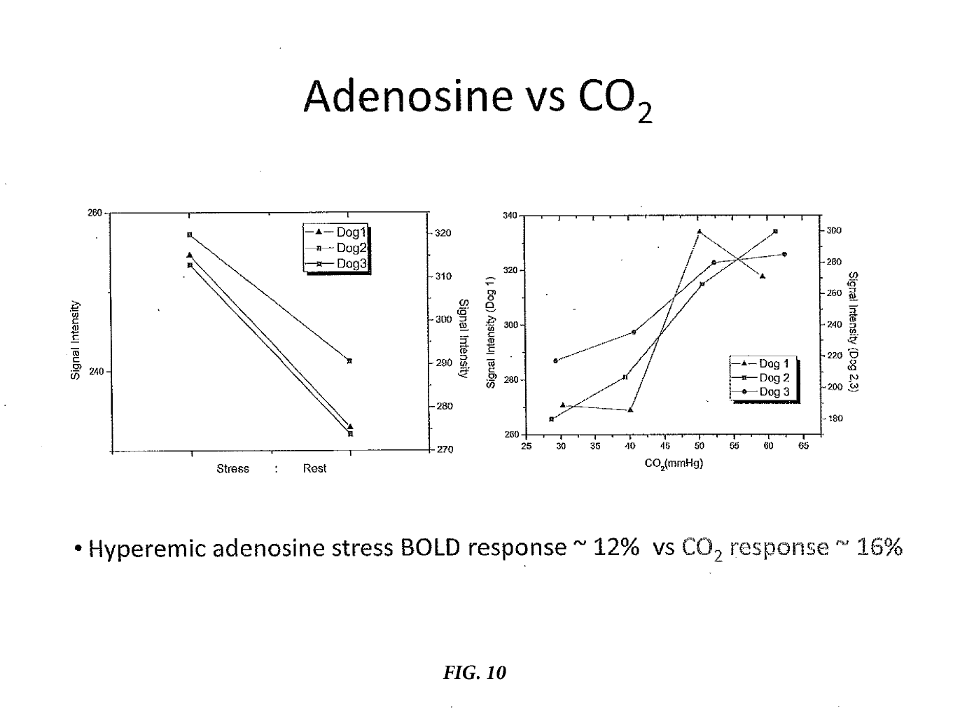

Vascular reactivity of three canines that were administered with adenosine was compared with the vascular reactivity of canines that were administered with CO₂. As shown in FIG. 10, the hyperemic adenosine stress BOLD response is approximately 12% compared with 16% in response to CO₂.

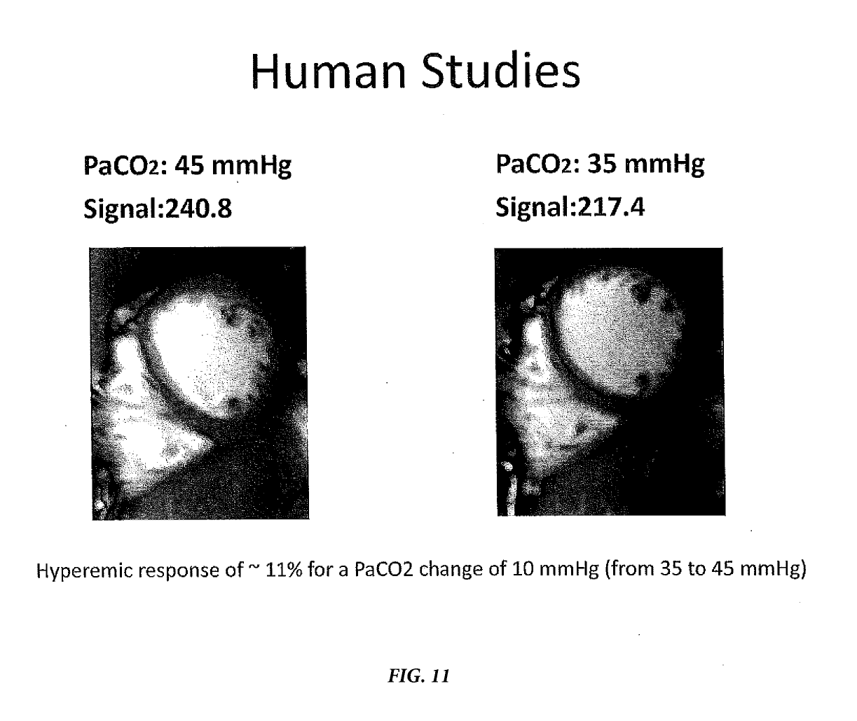

Further, as shown in FIG. 11, early human data shows a hyperemic response of approximately 11% for a partial pressure CO2 (pCO2) change of 10 mmHg, from 35 mmHg to 45 mmHg.

### Example 6

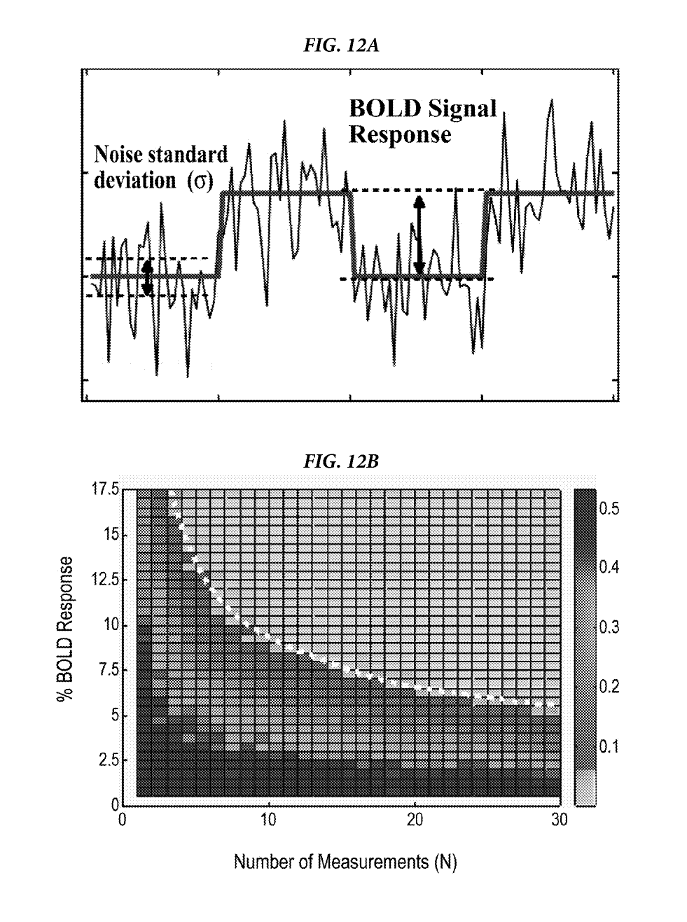

To derive a theoretical understanding of how repeated measurements may affect the BOLD signal response, for a given BOLD response to PaCO₂, Applicants performed numerical simulations of statistical fits assuming various peak hyperemic BOLD responses to two different PaCO₂ levels (as in FIG. 12a) along with known variability in BOLD signals. The results (FIG. 12b) showed that as the BOLD response decreases, the number of measurements required to establish statistical significance (p<0.05) associated with the BOLD response increases exponentially. This model provides the basis for developing a statistical framework for identifying ischemic volume on the basis of repeated measures.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of inducing hyperemia to diagnose coronary heart disease in a subject in need thereof comprising administering a CO₂ containing gas, attaining at least one increase in a subject's coronary PaCO₂ sufficient for diagnosing coronary heart disease from imaging data, and imaging the heart during a period in which the at least one increase in PaCO₂ is measurable to produce imaging data indicative of a cardiovascular-disease-associated vasoreactive response in at least one coronary blood vessel or region of the heart.

2. The method of claim 1, comprising attaining the at least one increase in PaCO₂ in a stepwise manner.

3. The method of claim 1, comprising attaining the at least one increase in PaCO₂ in a block manner.

4. The method of claim 1, comprising administering carbon dioxide via inhalation to attain a predetermined PaCO₂.

5. The method of claim 4, wherein the predetermined PaCO₂ is subject specific.

6. The method of claim 5, wherein the selected increase in PaCO₂ is an 8 to 20 mm Hg increase in a subject's steady state level measured prior to changing the subject's PaCO₂.

7. The method of claim 1, wherein the cardiovascular disease-associated vasoreactive response is a compromised increase in blood flow.

8. The method of claim 1, wherein the imaging method is PET or SPECT and the measure of the cardiovascular-disease-associated vasoreactive response is the presence or absence of a threshold increase in blood flow.

9. The method of claim 1, wherein the imaging data is indicative of the presence or absence of a two-fold increase in blood flow.

10. The method of claim 1, wherein the PaCO₂ is increased and decreased in a block manner repeatedly.

11. The method of claim 1, wherein the imaging data are obtained by MRI.

12. The method of claim 1, wherein the imaging data are a change in signal intensity of a BOLD MRI signal.

13. The method of claim 1, wherein the presence or absence of coronary heart disease is assessed on the basis of whether or not the at least one increase in PaCO₂ produces at least an 8%-20% increase in BOLD signal intensity.

14. The method of claim 1, wherein the presence or absence of coronary heart disease is assessed on the basis of whether or not the at least one increase in PaCO₂produces at least a 9%-12% increase in BOLD signal intensity.

15. The method of claim 1, wherein the presence or absence of coronary heart disease is assessed on the basis of whether or not the at least one increase in PaCO₂ produces at least a 10% increase in a BOLD MRI signal

16.-17. (canceled)

18. The method of claim 11, comprising (i) registering and segmenting MRI images to obtain the myocardial dynamic volume and (ii) identifying ischemic territory and quantifying image volume.

19. The method of claim 12, comprising (i) imaging the myocardium to obtain free-breathing cardiac phase resolved 3D myocardial BOLD images; (ii) registering and segmenting the images to obtain the myocardial dynamic volume; and (iii) identifying ischemic territory and quantifying image volume.

20.-22. (canceled)

23. A method for imaging hyperemia in a subject in need of a diagnostic assessment of cardiovascular disease comprising administering a CO₂ containing gas in a non-therapeutic diagnostic setting, attaining at least one selected increase in a subject's coronary PaCO₂ sufficient for diagnosing coronary heart disease from imaging data and imaging the heart during a period in which the selected increase in PaCO₂ is measurable, wherein the imaging data is selected to be indicative of a cardiovascular-disease-associated vasoreactive response in at least one coronary blood vessel or region of the heart.

24.-25. (canceled)

26. The method of claim 23, wherein the cardiovascular-disease-associated vasoreactive response is comparable to a vasodilatory response produced by administering a hyperemia inducing drug for a duration and in an amount per unit of time effective to assess coronary disease.

27.-29. (canceled)

30. The method of claim 23, wherein the cardiovascular-disease-associated vasoreactive response is obtained by controlling the administration of a CO₂ containing gas to repeatedly alternate between at least two PaCO₂ levels and obtaining repeat BOLD MRI measurement at each level to statistically assess the hyperemic response.

31.-35. (canceled)
